(19) United States
(12) Patent Application Publication
(10) Pub. No.: US 2012/0165768 A1
(43) Pub. Date: Jun. 28, 2012

(54) MALE URINE COLLECTOR

(76) Inventors: Mihoko Sekiyama, Kawasaki-shi (JP); Eiji Iwatsubo, Kasuya-gun (JP)

(21) Appl. No.: 13/404,559

(22) Filed: Feb. 24, 2012

Related U.S. Application Data
(63) Continuation-in-part of application No. PCT/JP2010/064400, filed on Aug. 25, 2010.

(30) Foreign Application Priority Data
Aug. 25, 2009 (JP) JP2009-193907
Oct. 12, 2011 (JP) JP2011-224549
Feb. 9, 2012 (JP) JP2012-025886

(51) Int. Cl. A61F 5/457; A61F 5/453

(52) U.S. Cl. 604/353; 604/349

(57) ABSTRACT

A male urine collector is provided. The bag comprises an elongate urine collecting bag sized to be worn on a lower part of a male user. The bag comprising a proximal end with a penis receiving opening for receiving a penis of a male user and, a distal end configured to be closed to collect urine discharged from the user's penis in the bag and to be selectively opened to discharge the collected urine from the bag. The male urine collector comprises at least one thin pliable opening ring disposed around and attached to the proximal end of the bag to open the penis receiving opening.

### RELATED APPLICATIONS

This application is a continuation-in-part of PCT/JP2010/064400 filed on Aug. 25, 2010, which claims priority to Japanese Application Nos. JP2009-193907 filed on Aug. 25, 2009, JP2011-224549 filed on Oct. 12, 2011, and JP2012-025886 filed on Feb. 9, 2012. The entire contents of these applications are incorporated herein by reference.

### BACKGROUND OF THE INVENTION

1. Field of the Invention

The present invention relates to urinary drainage devices for persons such as elderly persons who cannot urinate by themselves at will. More particularly, the present invention relates to male urine collectors.

2. Description of the Related Art

Diapers are commonly used as urinary drainage devices. Diapers, however, suffer from various problems, e.g. uncomfortable feeling in use, a rash developed in the area where a diaper is worn, troublesomeness in changing diapers, and high costs. Accordingly, there have been developed various urine collectors replacing diapers.

One type of male urine collector comprises a rubber sheath worn on the penis of a man and a hose connected to the distal end of the sheath to collect urine in a urine bottle prepared at the distal end of the hose (see, for example, JP1998192347A, JP198446519U and JP1985109617U). There has been another type of urine collector comprising a urine collecting bag attached to the penis and secured to the thigh so that the urine collector can be used even when the user is going out (see, for example, JPU3025019). In addition, various schemes have been devised to wear a urine collector on the penis (see, for example, GB Patent 2191095A).

### SUMMARY OF THE INVENTION

An object of the present invention is to provide a male urine collector which is easier to attach and detach than the conventional ones and which can be worn reliably and used even when the user is going out wearing it.

The present invention provides a male urine collector comprising an elongate urine collecting bag sized to be worn on a lower part of a male user. The bag comprises a proximal end with a penis receiving opening for receiving a penis of a male user, and a distal end configured to be closed to collect urine discharged from the user's penis in the bag and to be selectively opened to discharge the collected urine from the bag.

The male urine collector may further comprise at least one thin pliable opening ring secured to the proximal end of the bag. The proximal end has an edge portion bent radially outward and securely attached to the at least one thin ring. The at least one thin pliable opening ring may comprise a proximal thin ring and a distal thin ring. The proximal and distal thin rings being axially aligned with each other and securely sandwich the edge portion of the proximal end of the bag.

The male urine collector may comprising an inside sleeve having water absorbability. The inside sleeve is disposed inside the proximal end of the bag so that the inside sleeve is positioned around the penis inserted into the proximal end.

The urine collector may have a closure detachably attached to the distal end of the urine collecting bag to close the urine discharge opening.

The urine collector may comprise an underwear worn on a lower part of the user serving as a urine collector wearing member. The underwear comprises a bag support portion having an inside surface disposed on the lower part of the user to cover the penis, an outside surface opposite the inside surface, and a bag support opening extending from the inside surface through the outside surface, the bag support opening being configured to allow the urine collecting bag to pass through the bag support opening in a state that has been received the penis into the proximal end of the bag and the thin ring is positioned inside of the inside surface. A pouch may be attached to the outside surface of the bag support portion of the underwear, the pouch being configured to receive therein the urine collecting bag extending outside of the underwear through the bag support opening and allow at least the distal end of the urine collecting bag to extend outside from the pouch for discharge of the urine collected in the urine correcting bag.

The urine collector may further comprises a seal member comprising sponge material and configured to be disposed around the proximal end of the urine collecting bag into which the penis which has been inserted, the thin ring of the urine collecting bag being, in use, engaged with a proximal end of the seal member; and, a securing ring having a tubular portion configured to be fitted around the seal member to press the proximal end of the urine collecting bag against the penis, the securing ring having a bag exit opening at a distal end of the securing ring to allow the urine collecting bag to extend distally through the bag exit opening.

In another aspect, the present invention provides a male urine collector comprising an elongate urine collecting bag wherein the bag comprises pliable sheet material, wherein the bag comprises a proximal end with a penis receiving opening for receiving a penis of a male user to collect urine discharged from the penis within the urine collecting bag and, at least one thin pliable opening ring disposed around and attached to the proximal end of the bag to open the penis receiving opening.

Embodiments of the male urine collector according to the present invention will be explained below with reference to the accompanying drawings. In the explanation, like reference numerals used in various embodiments indicate like elements.

### BRIEF DESCRIPTION OF THE DRAWINGS

FIG. 1 shows a urine collecting bag constituting a male urine collector according to an embodiment of the present invention, the bag having been worn on the penis of a male user.

FIG. 2 shows the urine collecting bag in FIG. 1 with a seal member disposed around the proximal end of the urine collecting bag.

FIG. 3 shows the way in which a securing ring constituting the male urine collector is fitted around the seal member in FIG. 2.

FIG. 4 shows the urine collector upon completion of wearing on the penis.

FIG. 5 shows a man as seen from the front who wears the male urine collector according to the present invention by means of a urine collector wearing member.

FIG. 6 is a rear view of the securing ring.

FIG. 7 is a sectional side view of the securing ring in FIG. 6.

FIG. 8 is a front view of a securing ring according to another embodiment.

FIG. 9 is a sectional side view of the securing ring in FIG. 8.

FIG. 10 is a perspective view of an opening ring provided around the proximal end of the urine collecting bag to open the proximal end and used in the securing ring shown in FIG. 9.

FIG. 11 is a sectional side view of a securing ring according to still another embodiment.

FIG. 12 is a sectional side view of a urine collecting bag according to a further embodiment.

FIG. 13 is a drawing showing another type of urine collector wearing member as worn on a user.

FIG. 14 is a developed view of the urine collector wearing member in FIG. 13.

FIG. 15 shows the way in which the male urine collector according to the present invention is worn by using the urine collector wearing member shown in FIG. 14.

FIG. 16 is a sectional side view showing another mode of attaching the urine collecting bag with the securing ring in FIG. 9.

FIG. 17 is a sectional side view of a urine collector according to a still further embodiment of the present invention.

FIG. 18 is a perspective view of a urine collecting bag of the urine collector of FIG. 17.

FIG. 19 is a front view of a urine bag wearing member or underwear in the shape of briefs that constitutes the urine collector of FIG. 17.

FIG. 20 is a sectional side view of a pouch of the urine collector of FIG. 17 and adapted to receive and hold the portion of a urine collecting bag of the urine collector 17 which distally extends from the proximal end of the urine collecting bag.

FIG. 21 is a rear view of the pouch of FIG. 19.

FIG. 22 is a front view of the pouch of FIG. 19.

FIG. 23 is a modification of the underwear of FIG. 19.

FIG. 24 is a longitudinal sectional view of another type of urine collecting bag.

FIG. 25 is a perspective view of an inside sleeve of the urine collecting bag of FIG. 24.

FIG. 26 is a longitudinal sectional view of the inside sleeve of FIG. 25.

FIG. 27 is a longitudinal sectional view of a modification of the urine collecting bag FIG. 24.

FIG. 28 is a longitudinal sectional view of a bag blank that will be shaped into the urine bag shown in FIG. 27.

### DETAILED DESCRIPTION OF THE PREFERRED EMBODIMENTS

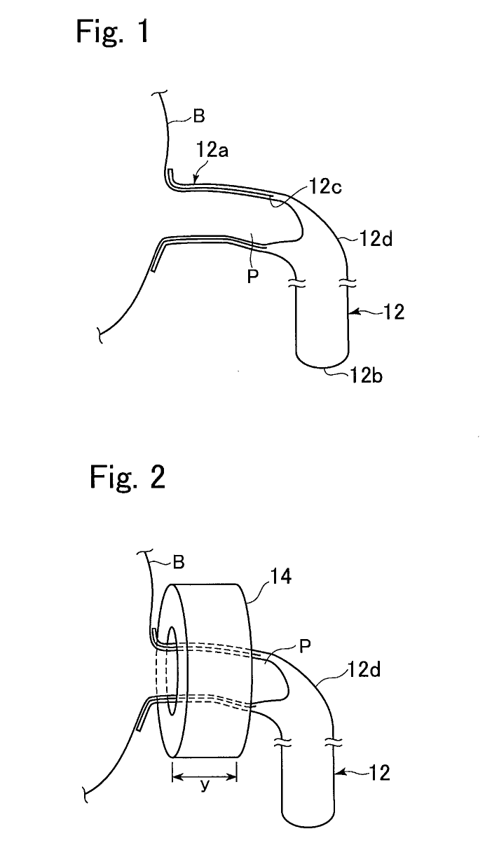

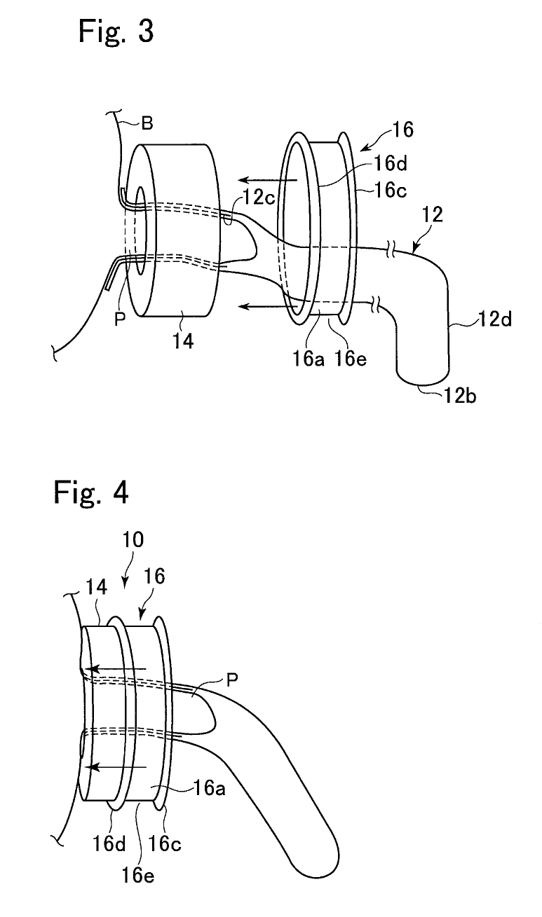

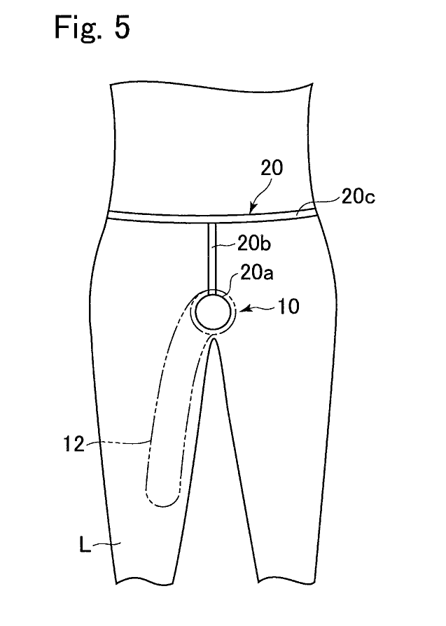

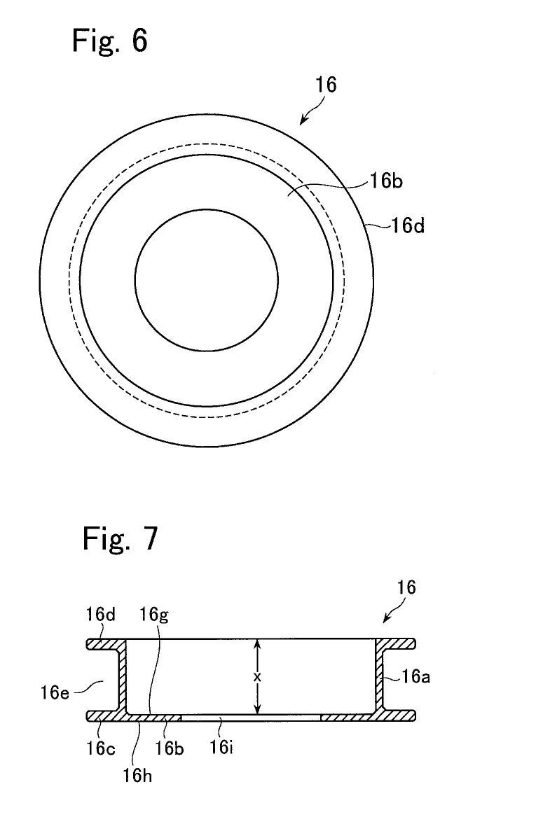

A first embodiment of a male urine collector 10 according to the present invention will be explained with reference to FIGS. 1 to 6, which show the procedure for wearing the same. The male urine collector 10 (FIG. 4) includes a urine collecting bag 12 (FIG. 1) made of a polyethylene sheet or the like. The urine collecting bag 12 has an openable proximal end 12a and a closed distal end 12b . The urine collecting bag 12 is usually collapsed with the proximal end closed prior to use and, when used, the proximal end 12a is opened to receive the penis P of a male user. The male urine collector 10 further includes a seal member 14 (FIG. 2) made of a sponge material (porous soft material) annularly fitted around the proximal end 12a of the urine collecting bag 12 which has received the penis P, and a securing ring 16 (FIGS. 3 and 4) fitted around the seal member 14 to press the seal member radially inward so as to secure the urine collecting bag 12 around and in close sealing contact with the penis P. Further, the male urine collector 10 includes a urine collector wearing member 20 (FIG. 5) that is adapted to be worn around the waist of the male user to hold the urine collecting bag with the seal member 14 and the securing ring 16 attached around the proximal end of the urine collecting bag.

The proximal end 12a of the bag 12 preferably has a folded back inward portion 12c. This is for prevention of urine stored in the bag from leaking out easily. As shown in FIG. 5, the urine collecting bag 12 may be set along one leg L of the user.

To set the urine collecting bag 12 along one leg of the user, for example, surface fastener members such as those commercially available as “Magic Tape (Registered Trademark)” from Kuraray may be provided, respectively, on the outer surface of the urine collecting bag 12 and on the inside of one leg of the user's pants, and the surface fastener member provided on the urine collecting bag 12 with the penis P received therein at the proximal end thereof is engaged with the surface fastener member provided on the inside of the pants to support the urine collecting bag on the inner surface of the pants. As other examples, a pouch for accommodating the urine collecting bag may be attached to the inner surface of the pants. Alternatively, an elongated strap may be securely attached to the outer peripheral surface of the urine collecting bag so as to be wound around the user's leg to secure the urine collecting bag on the leg.

The sponge material of the seal member 14 softly abuts against the user's belly surface B and penis P and allows air to remain around the belly surface B and the penis P. Therefore, the sponge material is unlikely to cause a rash or other problems on the skin and hence an excellent material. The seal member 14 is preferably formed in a split ring structure so that it can be adjustably wound on the penis according to the size thereof.

The securing ring 16 may be made of a plastic material or the like and has a tubular portion 16a (FIGS. 4, 6 and 7) set around the seal member 14 annularly worn around the penis P. The securing ring 16 further has an annular portion 16b provided along a distal end edge of the tubular portion 16a closer to the distal end of the penis P to extend radially inward, and annular flange portions 16c and 16d provided along two axially opposite end edges or distal and proximal end edges, respectively, of the tubular portion 16a to extend radially outward. The annular portion 16b has an annular interior surface 16g, an annular exterior surface 16h, and a radially inside axial surface extending between radially inside edges of the interior and exterior surfaces 16g, 16h to define a bag exit opening 16i. The flange portions 16c and 16d form a groove 16e therebetween for attaching the urine collector wearing member 20.

Preferably, the securing ring 16 has an axial length x that is substantially a half of the axial length y of the ring-shaped seal member 14. The securing ring 16 is fitted around the seal member 14 by putting the tubular portion 16a onto the seal member 14 from the distal end of the seal member 14 closer to the penis distal end. Substantially a half of the seal member 14 on the side thereof closer to the user's belly is out of the tubular portion 16a so as to abut against the user's belly surface B.

The urine collector wearing member 20 may be made of an elongated cloth-like member and comprises an annular portion 20a fitted in the groove 16e between the flange portions 16c and 16d of the securing ring 16. The urine collector wearing member 20 further comprises a suspender portion 20b comprising a string-shaped member extending upward from the annular portion 20a along the user's abdomen, and a waist belt portion 20c worn around the user's waist and connected with the suspender portion 20b to support the securing ring 16 through the suspender portion 20b.

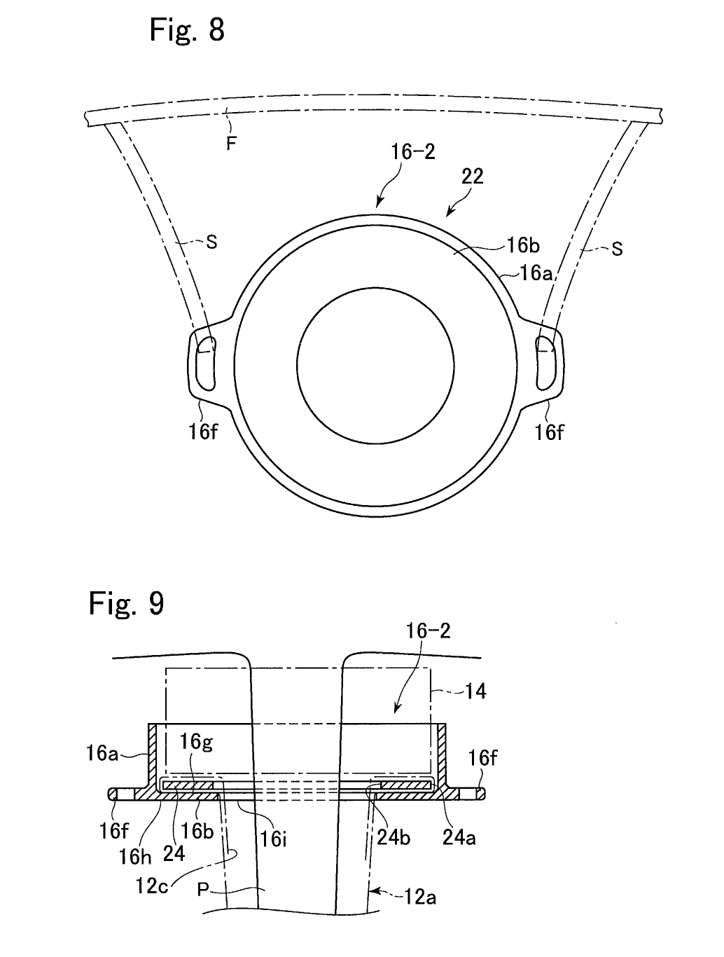

FIGS. 8 and 9 show a securing ring 16-2 according to another embodiment. The securing ring 16-2 has substantially the same structure as the above-descried securing ring 16. However, the securing ring 16-2 is not provided with the flange portions 16c and 16d, which are provided on the securing ring 16, but has a pair of left and right ear-shaped portions 16f provided on the distal end edge of the tubular portion 16a at diametrically opposite positions. In the securing ring 16-2, strips or strings S are attached to the ear-shaped portions 16f. The strings S are extended upward along both sides of the user's abdomen and connected to a waist belt portion F worn around the user's waist.

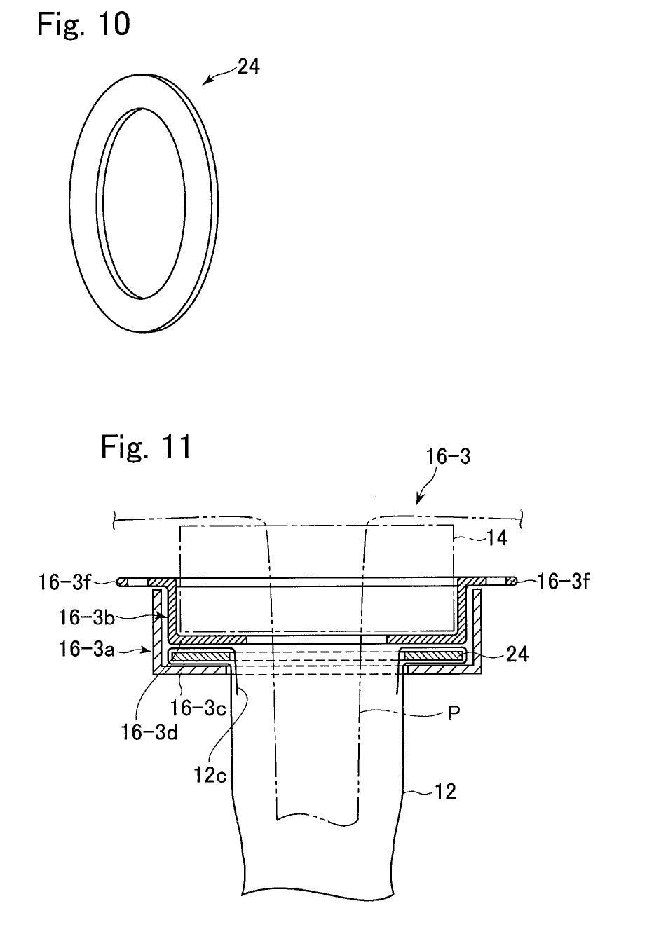

As shown in FIG. 9, the securing ring 16-2 uses a thin opening ring 24 as shown in FIG. 10 to attach the urine collecting bag 12 to the securing ring 16-2. The opening ring 24 is first put around the urine collecting bag 12 at the proximal end and the proximal end 12a is, then, folded radially inward from an outer peripheral edge 24a of the opening ring 24 and further extended away from the user's body from an inner peripheral edge 24b of the opening ring. The opening ring 24 with the proximal end 12a of the urine collecting bag 12 disposed therearound is put inside the securing ring 16-2 so as to be adjacent to the annular interior surface 16g of the annular portion 16b of the securing ring. Preferably, the opening ring 24 set in the above-described state may be securely fixed to the proximal end 12 of the bag 12 by thermowelding or the like. This enables the urine collecting bag 12 to be easily set inside the securing ring 16-2 and to be surely prevented from coming off the securing ring 16-2. In this embodiment, the seal member 14 is directly worn around the penis P.

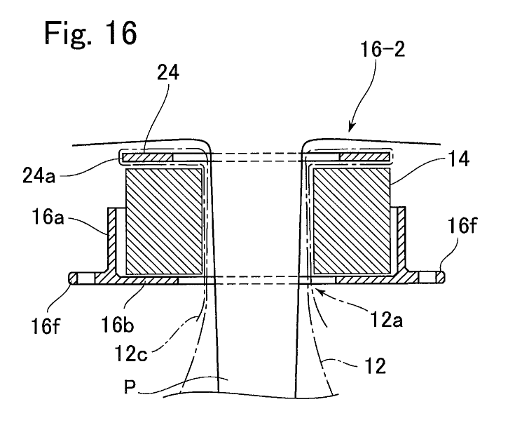

The urine collecting bag 12 with the opening ring 24 may be worn as shown in FIG. 16. In this case, the proximal end of the urine collecting bag 12 is fitted over the penis as far as the base or proximal end the penis, and the seal member 14 is fitted around the proximal end of the bag so that the opening ring 24 is set to be adjacent to the proximal end surface of the seal member 14. The securing ring 16 is fitted around the seal member 14 to press the seal member radially inward so as to secure the urine collecting bag 12 around and in sealing contact with the penis P. In the example shown in FIG. 16, the proximal end of the bag element has a fold back outside portion 12c so as to wrap the opening ring 24.

FIG. 11 shows a modification of the securing ring 16-2 shown in FIG. 9. The securing ring 16-3 comprises a first securing ring, or an inner ring 16-3b, and a second securing ring, or an outer ring 16-3a, which is fitted to the outside of the inner ring. The opening ring 24 attached to the proximal end 12a of the urine collecting bag 12 as stated with reference to FIG. 9 is held and secured between an annular potion 16-3c of the outer ring 16-3a and an annular portion 16-3d of the inner ring 16-3b. The securing ring 16-3 has ear-shaped portions 16-3f provided on the peripheral edge of the inner ring 16-3b, which are similar to the ear-shaped portions 16f of the securing ring shown in FIGS. 8 and 9. The outer ring 16-3a is connected to the inner ring 16b by a proper connecting system, preferably, for example, a bayonet system (i.e. a system in which the outer ring is axially fitted to the inner ring and thereafter rotated relative to the inner ring, thereby connecting the outer and inner rings to each other).

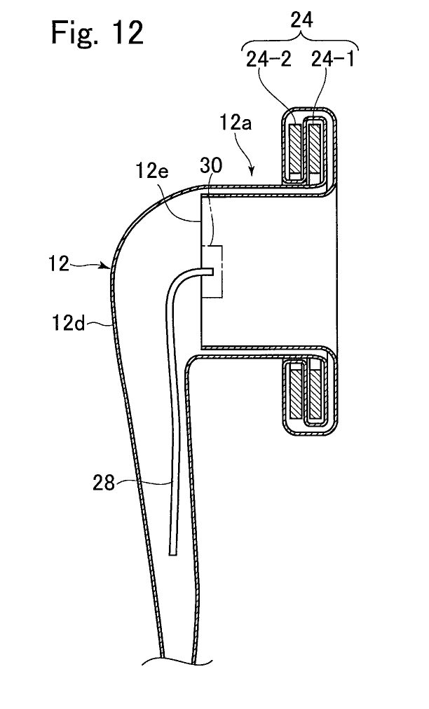

FIG. 12 shows a part of a male urine collector according to a further embodiment. In this embodiment, the opening ring 24 comprises first or proximal and second or distal rings 24-1 and 24-2 having substantially the same size and coaxially stacked over one another. The proximal end of the urine collecting bag 12 that has been proximally passed through the respective insides of the second and first rings 24-2 and 24-1 and folded back radially outward along the first ring 24-1. Further, the proximal end of the bag 12 is passed radially inward between the first and second rings 24-1 and 24-2 and distally passed through the inside of the second ring 24-2. Further, the proximal end of the bag 12 is folded back radially outward along the second ring 24-2 and bent to pass outside the second and first rings 24-2 and 24-1 before being folded back radially inward along the first ring 24-1 to distally extend toward the distal end of the urine collecting bag through the respective insides of the first and second rings 24-2 and 24-1.

The proximal end edge 12e of the bag 12 is provided with an elongated linear urine guide 28 secured thereto by an adhesive member 30, e.g. adhesive tape. The elongated urine guide 28 extends toward the distal end of the urine collecting bag 12 along the inner surface of the bag 12. As stated above, before use, the urine collecting bag is folded into a small shape and, therefore, the bag is beforehand flattened throughout the entire length of the bag. Accordingly, even when the urine collecting bag is attached to the penis of a user, the portion distally extending from the proximal end where the penis has been received may remain flattened and the inner walls of the distally extending portion adhere to each other. Accordingly, when urine is discharged from the penis, the discharged urine may be prevented from dropping toward the distal end of the bag. The urine guide is for preventing this problem. Specifically, urine discharged from the penis P is guided along the urine guide to run downward and to thereby separate the adhering bag inner walls and open the distal portion.

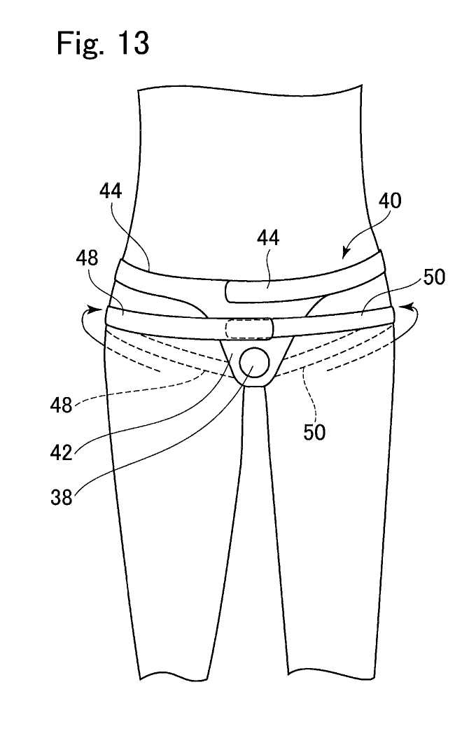

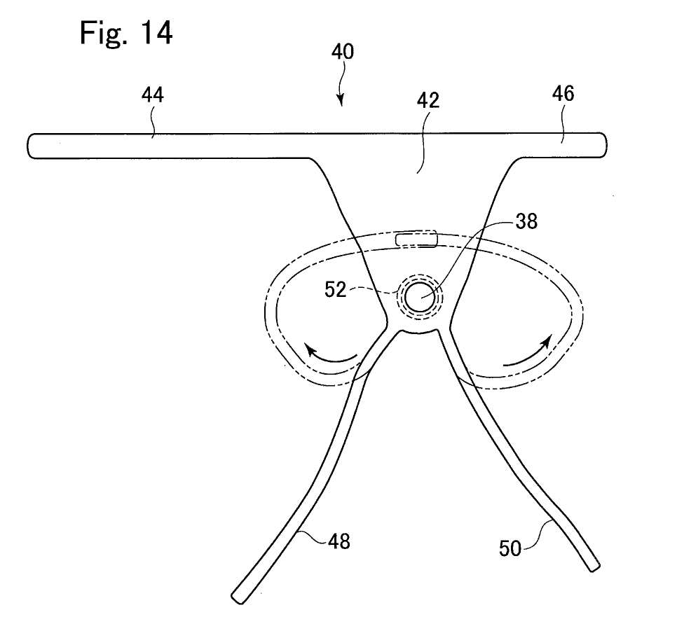

FIGS. 13 to 15 show an underwear serving as the urine collector wearing member 40 according to a further embodiment. The underwear or urine collector wearing member 40 has a substantially inverted triangular bag support portion 42 made of a cloth-like member and abutted against the center of the lower abdomen and further having a bag support opening 38 at a position corresponding to the penis P extending through the bag support portion 42 from the proximal side to the distal side of the bag support portion. The underwear 40 further has waist belt portions 44 and 46 extending from the opposite sides of the upper end of the bag support portion 42. In use, the waist belt portions 44 and 46 are passed around the user's torso or waist from the right and left, respectively, so that the waist belt portion 44 overlaps and adhereingly attached to the waist belt portion 46. In FIG. 13, the waist belt potion 44 extends over the waist belt portion 46 to the bag support portion 42 and adhereingly attached to the bag support portion 42 as well. Further, the urine collector wearing member 40 has auxiliary belt portions 48 and 50 extending right and left from the opposite sides of the lower end of the bag support portion 42. In use, the auxiliary belt portions 48 and 50 are extended rearward from the user's crotch and passed around the lower parts of the user's right and left buttocks (or the upper end parts of the user's right and left legs) to extend to the bag support portion 42 and then adheringly attached to the bag support portion 42 directly above the bag support opening 38. The waist belt portions 44 and 46 and the auxiliary belt portions 48 and 50 are provided with surface fasteners or the like for the adhering attachment. The auxiliary belt portion 48 and 50 will allow passage of solid waste or stool with the underwear worn as stated above.

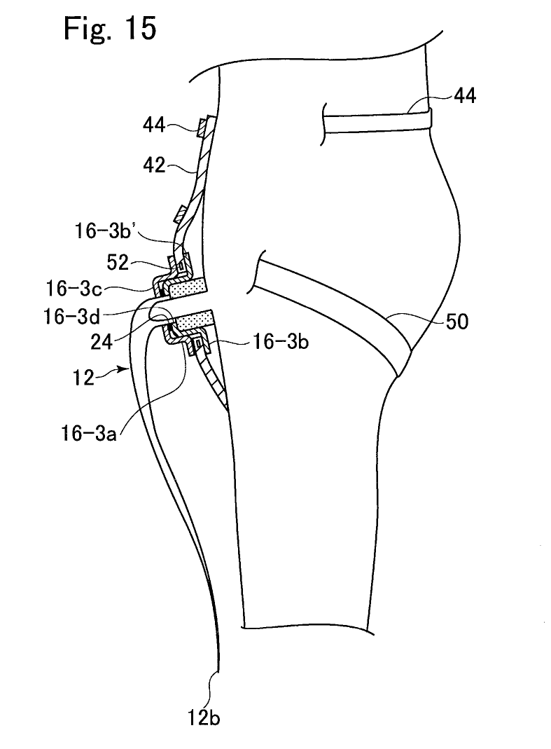

FIG. 15 shows an example in which the user uses the underwear or urine collector wearing member 40 to wears on his body a urine collecting bag 12 having a securing ring comprising outer and inner rings 16-3a and 16-3b in the same way as shown in FIG. 11. The inner ring 16-3b in this case has an annular wearing flange 16-3b′ along the rear or distal end edge thereof in place of the ear-shaped portions 16-3f in the embodiment shown in FIG. 11. The wearing flange 16-3b′ is set inside the bag support portion 42 of the underwear 40 , and the tubular portion of the inner ring 16-3b is set to extend outward through the bag support opening 38. The outer ring 16-3a has the tubular portion thereof fitted onto and secured to the tubular portion of the inner ring 16-3b. The urine collecting bag 12 has the opening ring 24 provided at the proximal end opening thereof The opening ring 24 is sandwiched between the annular potions 16-3c and 16-3d of the outer and inner rings 16-3a and 16-3b. In this state, the urine collecting bag 12 extends to the outside of the outer and inner rings 16-3a and 16-3b. The urine collector wearing member 40 has a retaining ring 52 attached along the circumference of the bag support opening 38 to prevent the annular wearing flange 16-3b′ of the inner ring 16-3b from coming off through the bag support opening 38. The retaining ring 52 is formed of a relatively rigid resin or the like. This male urine collector enables the urine collecting bag to be attached and detached by attaching and detaching the outer ring to and from the inner ring.

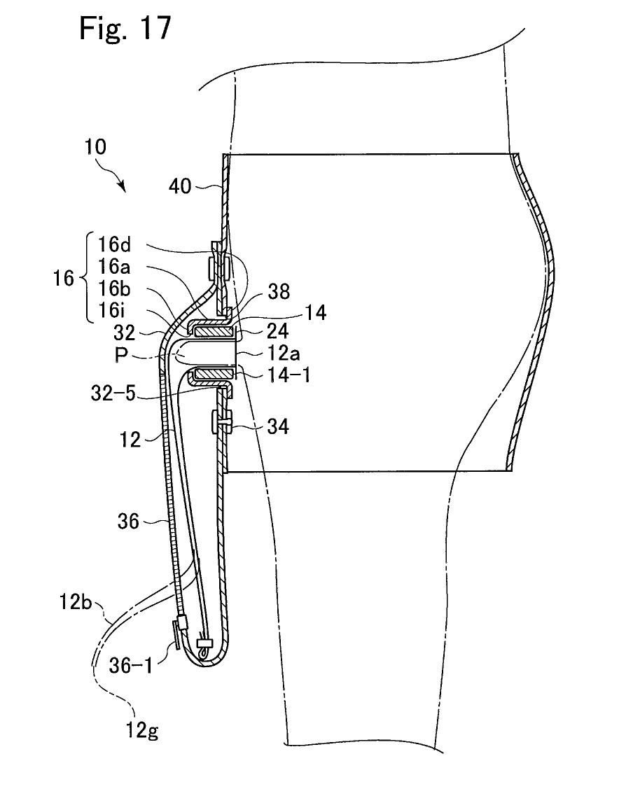

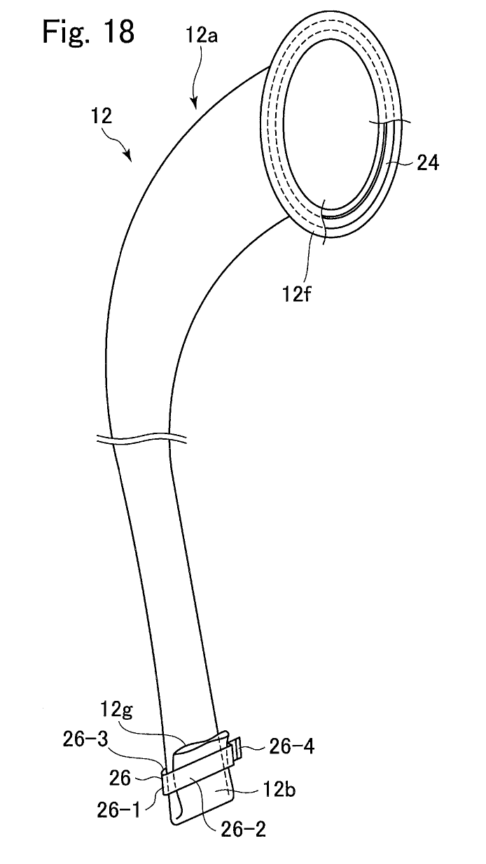

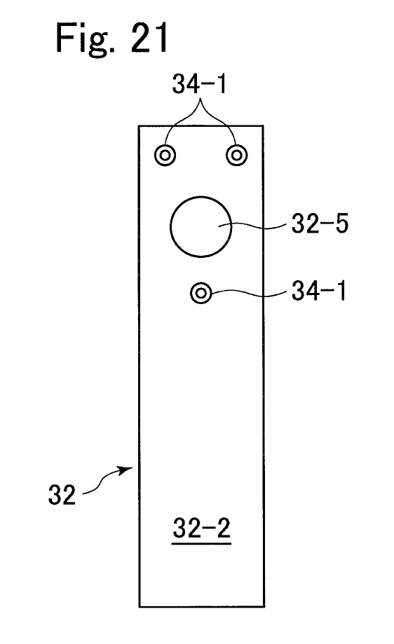

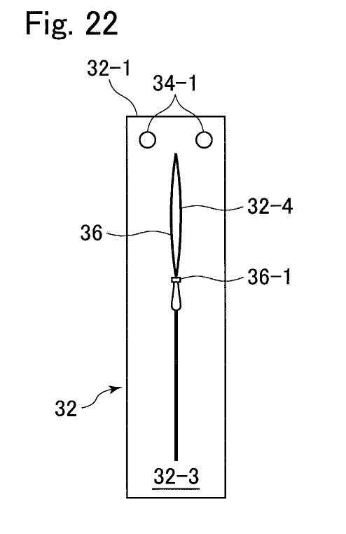

FIGS. 17-23 show a male urine collector 10 according to still another embodiment of the present invention. The male urine collector 10 includes a urine collecting bag 12 as best shown in FIG. 18. The proximal end 12a of the urine collecting bag 12 has an edge portion 12f bent radially outward and a thin opening ring 24 wrapped by and securely attached to the edge portion 12f thereof. The distal end 12b of the urine collecting bag 12 is configured to be selectively closed to collect or store urine discharged from the user's penis in the bag 12 in the bag and to be selectively opened to discharge the collected urine from the bag 12. Specifically, the distal end 12b of the bag 12 has an opening 12g for discharging urine out of the bag 12 and adapted to be folded back thereon and clamped by a clip 26 to sealingly close the urine discharge opening 12g. The clip 26 is made of a metal strip or relatively rigid plastic strip and has a pair of clamping arm portions 26-2 and 26-3 hingedly connected by a hinge portion 26-1 so that the clamping arms portions 26-2 and 26-3 clamp the folded back distal end 12b therebetween and detachably fastened each other by a fastening means 26-4 thereby closing the urine discharging opening 12g.

As in the urine collector shown in FIG. 16, a seal member 14 made from sponge material is fitted around the proximal end 12a that has received the penis P with the opening ring 24 positioned between the proximal end of the seal member 14 and the lower abdomen of the user (FIG. 17). Around the seal member 14 is fitted a securing ring 16 to press the seal member 14 against the proximal end 12a of the urine collecting bag 12 thereby sealingly engaging the proximal end 12a with the penis P. As shown in FIG. 17, the securing ring 16 has a tubular portion 16a, a distal annular portion 16b extending radially inward from the distal end edge of the tubular portion 16a and a proximal annular portion 16d extending radially outward from the proximal end edge of the tubular portion 16a. The distal annular portion 16b defines an opening 32 for passing the urine collecting bag 12 forward out of the securing ring 16.

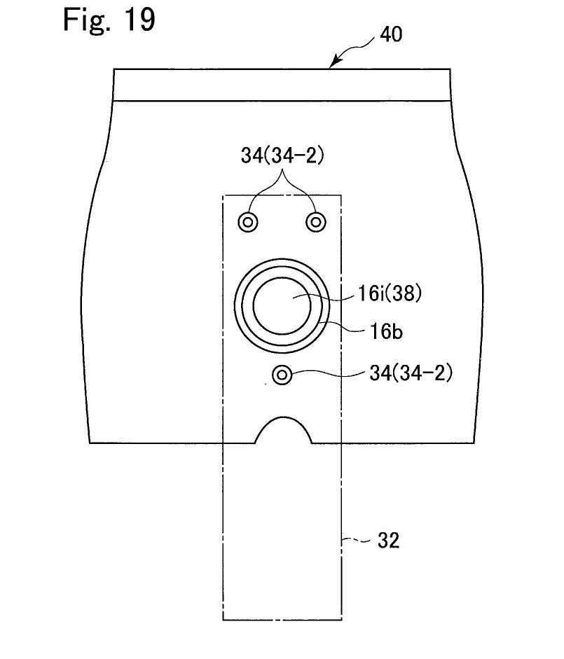

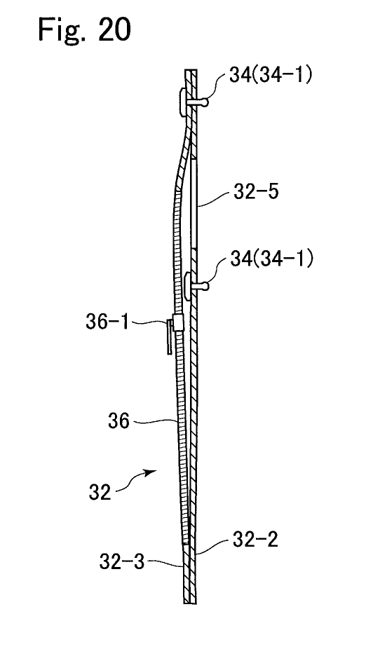

The urine collector 10 of this embodiment has an underwear 40 (FIG. 19) in the shape of briefs that serves as a urine collector wearing member and a pouch 32 provided on a front side (which may be referred to as a bag support part) of the underwear 40. The underwear 40 has a bag support opening 38 at a position corresponding to the penis P. The pouch 32 has a front part 32-3 and a rear part 32-2 (FIG. 20) that are both in the shape of a rectangular of same size and the circumferential edges thereof connected or stitched to each other. The rear part 32-2 of the pouch 32 has an opening 32-5 for passing the securing ring 16 fixed around the penis P so that the urine collecting bag 12 extending out from the securing ring 16 is receiving within the pouch 32. The front part 32-3 of the pouch 32 has a vertical slit 32-4 along which a zip fastener 36 is provided. The zip fastener 36 is open and closed by sliding a sliding tab 36-1 vertically so that, as shown in FIG. 17, at least the distal end 12b of the urine collecting bag 12 is pulled outside of the pouch to discharge the urine collected or stored in the bag 12 into a lavatory basin.

The pouch 32 is detachably attached to the front side of the underwear 40 by a plurality of hooks 34 each including a male hook member 34-1 and a female hook member 34-2. In this embodiment, the pouch 32 has two male hook members 34-1 adjacent the upper edge 32-1 of the pouch 32 about the opening 32-5 and one male hook member 34-1 directly below the opening 32-5 and the front side of the underwear 40 has corresponding three female hook members 34-2.

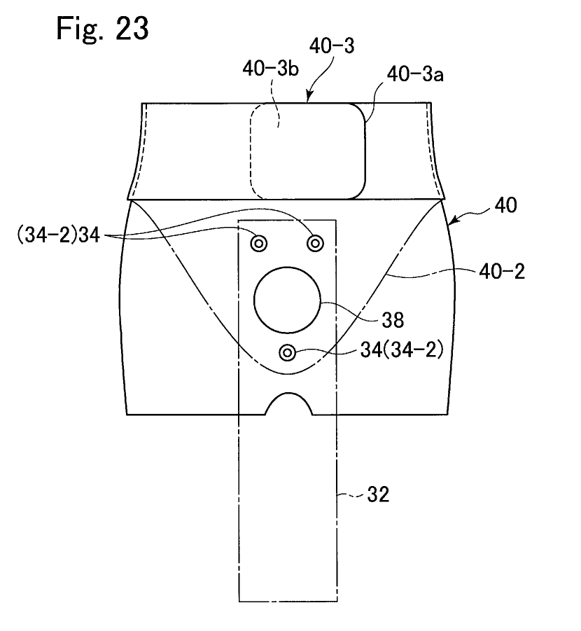

FIG. 23 shows a modification of the underwear 40. The modified underwear 40 includes a fastening belt 40-3 that is fastened around the waist portion of the underwear 40 in the shape of briefs. The fastening belt 40-3 has opposite ends 40-3a, 40-3b with surface fasteners (not shown) and is adapted to be tightly surrounds the waist portion of the underwear 40 and fastened by engaging the surface fasteners of the opposite ends 40-3a, 40-3b each other. The underwear 40 is provide on the interior surface thereof with a reinforcement cloth 40-2 which cooperates with the fastening belt 40-3 to positively support the urine collecting bag 12 on the user's body.

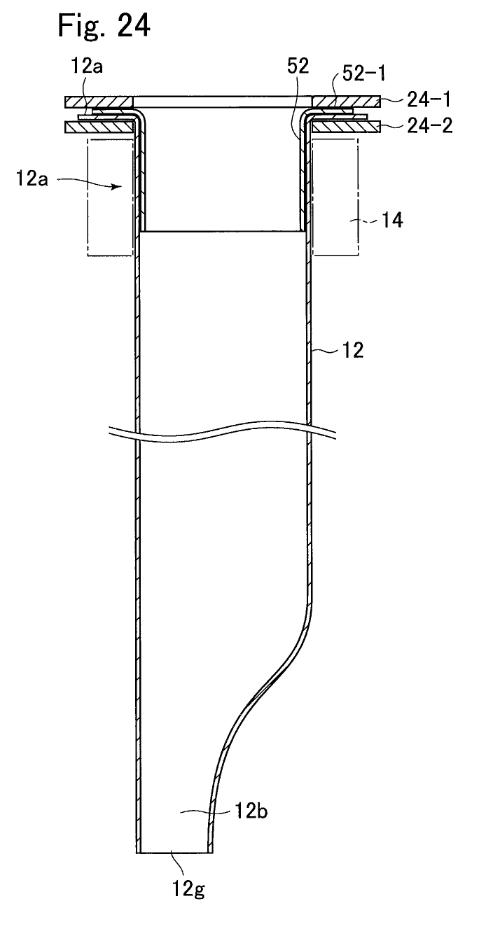

FIG. 24 shows further another embodiment of the urine collecting bag 12. The urine collecting bag 12 of this embodiment includes an inside sleeve 52 disposed inside the proximal end of the urine collecting bag 12. The inside sleeve 52 is made of material having water absorbability such as cloth and paper. The inside sleeve 52 is, as shown in FIG. 26, preferably provided on the interior surface 52-2 thereof with a water-shedding coating 54 such as polypropylene coating to keep the penis contacting the inside of the urine collecting bag dry as much as possible.

The inside sleeve 12 has a radially outward bent upper edge portion 52-1. The upper edge portion 52-1 is provided with a plurality of slits 52-1a that are circumferentially spaced apart from each other and extend radially to divide the upper edge portion 52-1 into a plurality of segments 52-1b in order to make it easy to bend the upper edge portion 52-1 radially outward. As shown in FIG. 24, the urine collecting bag has a pair of thin opening rings including a proximal opening ring 24-1 and a distal opening ring 24-2. The upper edge portion 52-1 of the inside sleeve 52 and the distal end edge portion 12f of the urine collecting bag 12 are sandwiched by the proximal and distal opening rings 24-1, 24-2 and securely attached to them by thermowelding, adhesive or the like. The proximal and distal opening rings 24-1, 24-2 are made of thin and pliable material such as paper and cloth so that the rings 24-1, 24-2 do not harm wearing comfortability of the urine collecting bag. For example, the distal opening ring 24-2 may be made of water-proof paper used paper used to fabricate a milk carton and the proximal opening ring 24-1 may be made of water-absorbable paper.

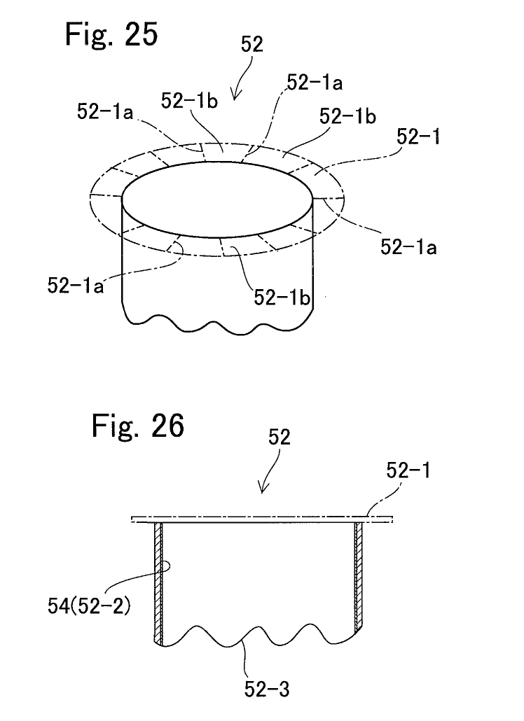

The inside sleeve 52 may have an indented distal edge 52-3. The edge shown in FIGS. 25 and 26 are in the shape of a wave. The edge 52-3 may be in the shape of saw teeth, for example. This indented shape promotes dropping of water from the inside sleeve toward distal end of the urine collecting bag.

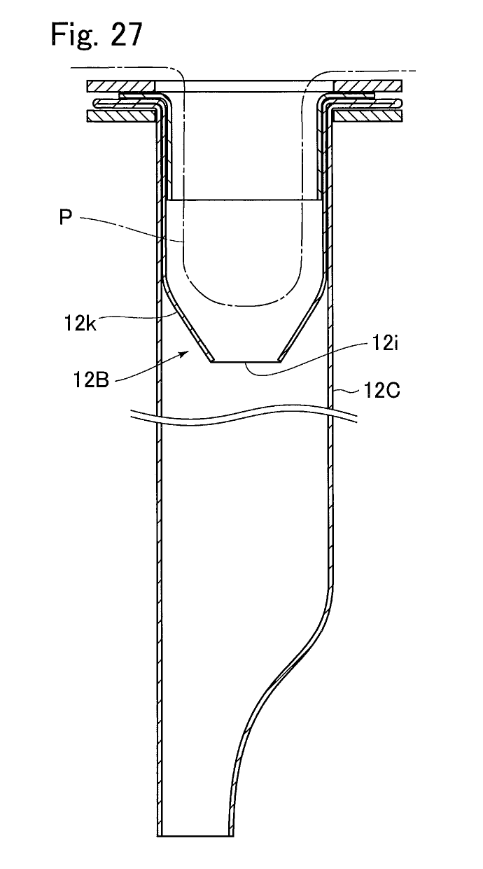

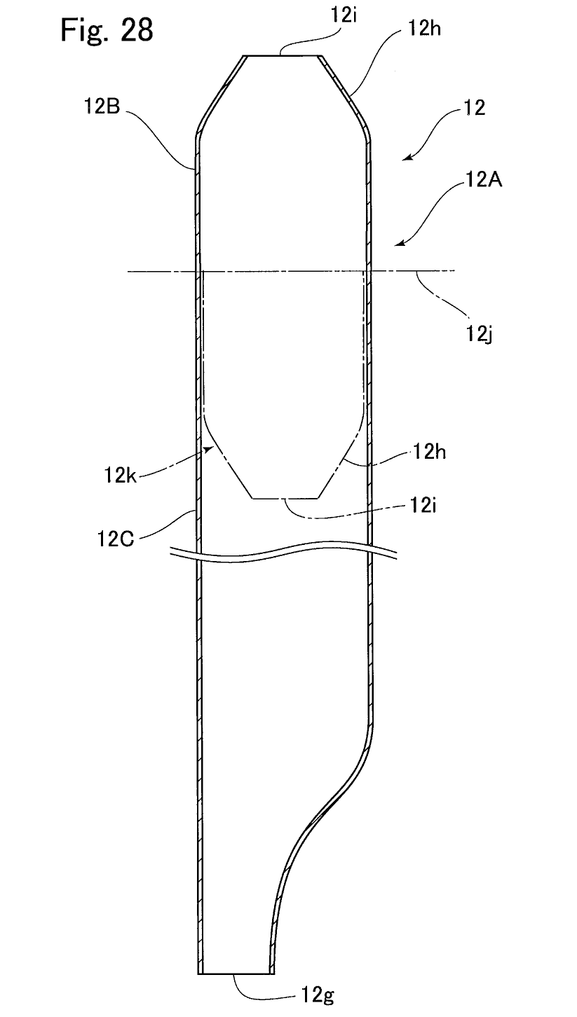

FIGS. 27 and 28 show a further embodiment of the urine collecting bag 12. FIG. 28 shows a blank 12A of the urine collecting bag 12 which has not yet been shaped into a shape as shown in FIG. 27. The bag blank 12a is generally in the shape of a tube having an upper tapered end 12h with an opening 12i and a lower end 12b with a urine discharging opening 12g. The upper part 12B of the bag blank 12A is folded back along around a line 12j into the lower part 12C of the bag blank 12 so that the bag blank 12A is finally shaped into the urine collecting bag 12 shown in FIG. 27. The urine collecting bag 12 has a check valve portion 12k formed by the folded back upper portion 12B wherein urine discharged from the penis P is permitted to flow down through the opening 12i whereas the urine collected into the lower part 12C is prevented from entering into the check valve portion 12K through the opening 12i.

Although some embodiments of the male urine collector according to the present invention have been explained above, the present invention is not limited to the foregoing embodiments but can be modified in a variety of ways. For example, the urine collector wearing member 20 that allows the urine collector to be supported by the user's body may be configured to be suspended from the user's neck.

What is claimed is:

1. A male urine collector comprising an elongate urine collecting bag sized to be worn on a lower part of a male user, the bag comprising:
a proximal end with a penis receiving opening for receiving a penis of a male user; and,
a distal end configured to be closed to collect urine discharged from the user's penis in the bag and to be selectively opened to discharge the collected urine from the bag.

2. The male urine collector of claim 1, further comprising at least one thin pliable opening ring disposed around the proximal end of the bag and extending radially outwardly, wherein the proximal end has an edge portion bent radially outward and securely attached to the at least one thin pliable opening ring.

3. The male urine collector of claim 1, further comprising an inside sleeve having water absorbability, the inside sleeve being disposed inside the proximal end of the bag so that the inside sleeve is positioned around the penis inserted into the proximal end.

4. The male urine collector of claim 3, wherein the inside sleeve comprises one of cloth and paper.

5. The male urine collector of claim 3, wherein the inside sleeve has an exterior surface including a radially outside surface and a radially inside surface, the exterior surface being at least partially of water-shedding.

6. The male urine collector of claim 3, wherein the radially inside surface is of water-shedding.

7. The male urine collector of claim 2,
wherein the at least one thin pliable opening ring comprises a proximal thin pliable ring and a distal thin pliable ring; and,
wherein the proximal and distal thin rings being axially aligned with each other and securely sandwich the edge portion of the proximal end of the bag.

8. The male urine collector of claim 3, wherein the inside sleeve has a proximal edge and a distal edge and the distal edge is in an indented shape.

9. The male urine collector of claim 1, wherein the urine discharge opening has a smaller opening area than the penis receiving opening.

10. The male urine collector of claim 1, further comprising a closure detachably attached to the distal end of the urine collecting bag to close the urine discharge opening

11. The male urine collector of claim 1, further comprises:
an underwear worn on a lower part of the user, the underwear comprising a bag support portion having an inside surface facing a lower abdomen of the user to cover the penis, an outside surface opposite the inside surface, and a bag support opening extending from the inside surface through the outside surface, the bag support opening being configured to allow the urine collecting bag to pass through the bag support opening in a state that the penis has been received into the proximal end of the urine collecting bag and the thin pliable opening ring is positioned inside of the inside surface of the bag support portion of the underwear.

12. The male urine collector of claim 11, wherein the underwear comprises a waist band portion connected to the bag support portion and fixed around a waist of the user to hold the bag support portion covering the penis,

13. The male urine collector of claim 12, wherein the underwear comprises right and left auxiliary band portions extending from a lower end of the bag support portion, the right and left auxiliary band portions being, in use, extended from a crotch of the user and passed around lower parts of user's right and left buttocks to extend to the bag support portion and then attached to the bag support portion above the bag support opening.

14. The male urine collector of claim 11, further comprising a pouch attached to the outside surface of the bag support portion of the underwear, the pouch being configured to receive therein the urine collecting bag extending outside of the underwear through the bag support opening and allow at least the distal end of the urine collecting bag to extend out from the pouch for discharge of urine collected in the urine correcting bag.

15. The male urine collector of claim 1, further comprising:
a seal member comprising sponge material and configured to be fitted around the proximal end of the urine collecting bag into which the penis has been inserted, the thin pliable opening ring of the urine collecting bag being, in use, engaged with a proximal end of the seal member; and,
a securing ring having a tubular portion configured to be fitted around the seal member to press the proximal end of the urine collecting bag against the penis, the securing ring having a bag exit opening at a distal end of the securing ring to allow the urine collecting bag to extend distally through the bag exit opening.

16. The male urine collector of claim 15, wherein the securing ring comprises an annular portion extending radially inward from a distal end edge of the tubular portion and the bag exit opening is defined by the annular portion.

17. The male urine collector of claim 11, further comprising:
a seal member comprises sponge material and configured to be fitted around the proximal end of the urine collecting bag into which the penis has been inserted, the thin pliable opening ring of the urine collecting bag being, in use, engaged with a proximal end of the seal member; and,
a securing ring having a tubular portion configured to be fitted around the seal member to press the proximal end of the urine collecting bag against the penis and configured to pass through the bag support opening of the underwear, the securing ring having a bag exit opening at a distal end of the securing ring to allow the urine collecting bag to extend distally through the bag exit opening, the securing ring further comprising a distal annular portion extending radially inward from a distal end edge of the tubular portion and a proximal annular portion extending radially outward from a proximal end edge of the tubular portion, the bag exit opening being defined by the distal annular portion, the proximal annular portion being, in use, engaged with the inside surface of the under wear to prevent the securing ring from distally moving and disengaging from the underwear, the distal annular portion being, in use, engaged with the seal member disposed in the securing ring to prevent the seal member from distally moving out of the securing ring.

18. The male urine collector of claim 1, wherein the urine collecting bag has an elongated urine guide disposed inside the urine collecting bag such that the elongated urine guide distally extends from adjacent the penis inserted in the urine collecting bag to guide urine discharged from the penis toward the distal end of the urine collecting bag.

19. The male urine collector of claim 17, further comprising an additional outside securing ring configured to be detachably coaxially fitted over the securing ring, the additional outside securing ring comprising a distal end having an opening to allow the urine collecting bag to distally pass therethrough and a proximal end configured to cooperate with the proximal annular portion of the secure ring to clamp therebetween a circular edge portion of the bag support porting defining the bag support opening

20. A male urine collector of claim 1, wherein:
the urine collecting bag comprises an elongated tubular blank made of thin pliable sheet material, the elongated tubular blank having a proximal end portion with a proximal end opening and a distal end portion with a distal end opening, the proximal end portion being folded back into the elongated tubular blank to form the proximal end of the urine collecting bag having a doubled wall structure comprising the folded back proximal end portion and a portion of the elongated tubular blank outside the folded back proximal end portion, the proximal end opening of the folded back proximal end portion being positioned distally of the penis inserted into the proximal end of the urine collecting bag and having an open area smaller than that of the portion of the elongated tubular blank outside the folded back proximal end portion so that the proximal end opening allows free passage of urine discharged from the penis distally through the proximal end opening but prevents reverse passage of the urine through the proximal end opening, the distal end opening forming the urine discharge opening of the urine collecting bag.

21. A male urine collector comprising an elongate urine collecting bag:
wherein the bag comprises pliable sheet material;
wherein the bag comprises:
a proximal end with a penis receiving opening for receiving a penis of a male user to collect urine discharged from the penis within the urine collecting bag; and,
at least one thin pliable opening ring disposed around and attached to the proximal end of the bag to open the penis receiving opening.
